(19) United States
(12) Patent Application Publication
(10) Pub. No.: US 2015/0283308 A1
(43) Pub. Date: Oct. 8, 2015

(54) ENDOLUMINAL DEVICE AND METHOD OF IMPLANTING SAME

(71) Applicant: COOK BIOTECH, INCORPORATED, West Lafayette, IN (US)

(72) Inventor: Michelle Chutka, West Lafayette, IN (US)

(21) Appl. No.: 14/625,076

(22) Filed: Feb. 18, 2015

Related U.S. Application Data
(60) Provisional application No. 61/974,573, filed on Apr. 3, 2014.

(51) Int. Cl. A61L 31/16; A61L 31/08; A61L 31/14; A61F 2/86

(52) U.S. Cl. CPC A61L 31/16; A61F 2/86; A61L 31/08; A61L 31/148; A61F 2210/0014; A61F 2210/0076; A61F 2210/0004; A61L 2430/34

(57) ABSTRACT

Implanting an endoluminal device in a patient includes advancing the device to a treatment location in a patient, and deploying the device such that it contacts body tissue forming an inner wall of a body lumen. The implantation further includes inciting an immune reaction in the body tissue via a sclerosant in a coating of the device, and promoting ingrowth of new body tissue triggered by the sclerosant about the device via a bioremodelable material in the coating so as to anchor the device at the treatment location.

### TECHNICAL FIELD

The present disclosure relates generally to designs and techniques for anchoring an endoluminal device in a body lumen in a patient, and more particularly to anchoring an endoluminal device without the necessity of mechanical force, debridement or other fixation methods.

### BACKGROUND

A great many different devices are implanted into the human body in modern interventional medicine. Stents, grafts, filters and occlusion devices, for instance, are well known and widely used. Such devices may be implanted with the expectation that they will later be removed, for example vena cava filters placed following surgery or another form of treatment. Other implanted devices may be placed within a patient with no expectation that they will ever be removed. In either case, it is typically desirable to provide for some mechanism of retaining the implanted device at a desired treatment location.

Many stents have sufficient springiness that they can be held in relatively mild compression within a body lumen, and the natural tendency for the device to spring outwardly biases the device into contact with body tissue and thus provides resistance against dislodging. Barbs and the like are commonly used in stents to assist in or to enable retention. Various other implanted devices and materials may be sutured, stapled or retained by other mechanical contrivances. Preparation of the body tissue by debridement in an area where a device is to be implanted is also sometimes performed. Retention of implanted devices thus often requires either specialized design of the device to be implanted, or laborious and potentially technically challenging techniques performed during open surgery or peripheral intervention.

### SUMMARY OF THE DISCLOSURE

In one aspect, a method of implanting an endoluminal device in a patient includes advancing the endoluminal device through a body lumen in the patient to a treatment location, and deploying the endoluminal device at the treatment location such that the endoluminal device contacts body tissue forming an inner wall of the body lumen. The method further includes inciting an immune reaction in the body tissue via a sclerosant in a coating of the endoluminal device so as to trigger growth of new body tissue at the treatment location. The method still further includes promoting ingrowth of the new body tissue about the endoluminal device via a bioremodelable material in the coating, such that the device is anchored by the new body tissue at the treatment location.

In another aspect, an implantable endoluminal device includes a frame having a first frame end and a second frame end, and defining a longitudinal axis extending between the first frame end and the second frame end. The frame further includes an outer surface, and a coating upon the outer surface having an axial extent upon the frame less than a full axial length from the first frame end to the second frame end. The coating includes a sclerosant in a concentration sufficient to incite immune reaction in body tissue so as to trigger new body tissue growth, and a bioremodelable material for promoting ingrowth of the new body tissue about the frame so as to anchor the endoluminal device within a body lumen in a patient.

### BRIEF DESCRIPTION OF THE DRAWINGS

FIG. 1 is a side diagrammatic view, including a detailed enlargement, of an endoluminal device according to one embodiment;

FIG. 2 is a perspective view of an endoluminal device and delivery mechanism at one stage of positioning within a patient, according to one embodiment;

FIG. 3 is a partially sectioned side diagrammatic view of an endoluminal device positioned for service within a patient, according to one embodiment; and

FIG. 4 is a sectioned view taken along line 4-4 of FIG. 3 and diagrammatically showing the endoluminal device as it might appear first positioned within a patient on the left side of the drawing, and after residence within the patient for some time on the right side of the drawing.

### DETAILED DESCRIPTION

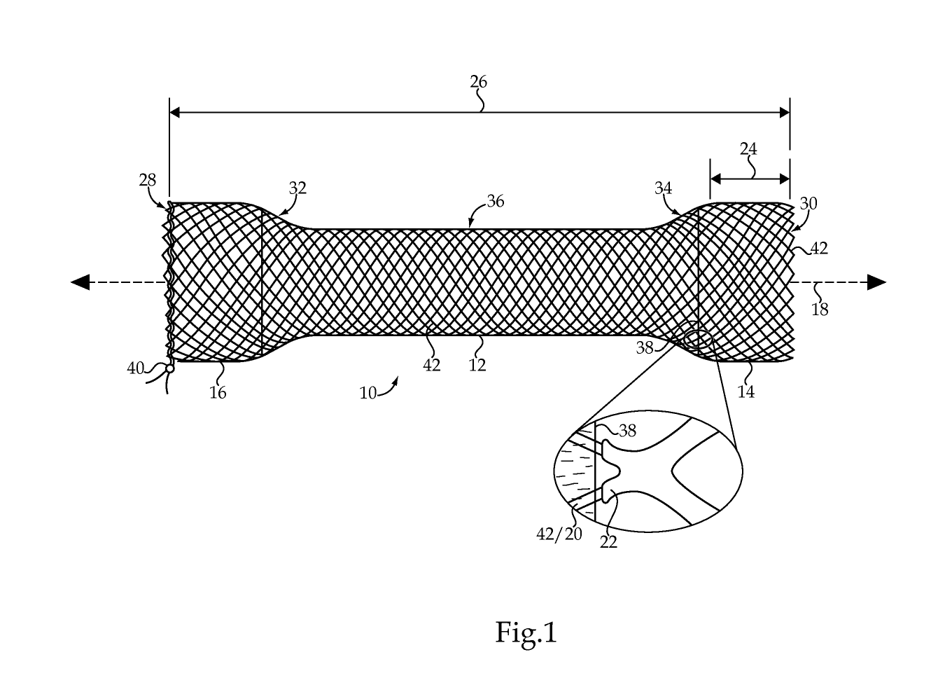

Referring to FIG. 1, there is shown an implantable endoluminal device 10 according to one embodiment, and including a frame 12 having a first frame end 14 and a second frame end 16. Frame 12 defines a longitudinal axis 18 extending between first frame end 12 and second frame end 14. Frame 12 also includes a proximal terminal end 28, and a distal terminal end 30. A proximal taper 32 transitions between second frame end 16 and a middle section 36 of frame 12. A distal taper 34 transitions between first frame end 14 and middle section 36. In the embodiment shown, frame 12 will be understood to have a generally cylindrical shape about first frame end 14, second frame end 16, and middle section 36, whereas tapers 32 and 34 are generally conical in shape. A silicone covering 38 is shown upon frame 12, and extends for a majority of an axial length 26 of frame 12, such that silicone covering 38 covers middle section 36 and each of tapers 32 and 34. In alternative embodiments, covering 38 might be omitted from the design, or modified to cover relatively more or less of the full axial length 26 of frame 12. The geometry of device 10 might also be modified such that middle section 36 has a diameter similar to that of first and second frame ends 14 and 16, or for certain applications a diameter greater than either of first and second frame ends 14 and 16. Many other design variations are possible within the scope of the present disclosure. In a practical implementation strategy, device 10 may include a stent of a type suitable for deployment within a gastrointestinal tract of a patient, although the present disclosure is not thereby limited. Grafts, occluders and filters, for instance, deployed anywhere in the body might be designed and used as contemplated herein. As will be further apparent from the following description, device 10 may be uniquely configured for anchoring within a body lumen of a patient without any necessity of mechanical force or spring biasing, debridement of the patient's body tissue, or other fixation methods such as spicules, barbs, sutures, or staples.

To this end, frame 12 may further have an outer surface 20, and a coating 22 upon outer surface 20 for use in anchoring device 10 at a treatment location. Coating 22 may have an axial extent 24 upon frame 12 that is less than full axial length 26 from terminal end 30 to terminal end 28. In a practical implementation strategy, only one end of frame 12 may be coated with coating 22, with axial extent 24 comprising about 25% or less, and more particularly about 10% or less, of full axial length 26. In other embodiments, a middle portion, such as part or all of middle section 36 might be coated with coating 22 to the exclusion of first and second frame ends 14 and 16, which might be left bare. In a practical implementation strategy, coating 22 will extend circumferentially around longitudinal axis 18 such that a generally cylindrical pattern of coating upon frame 12 is realized. Frame 12 might be dipped in liquid material or sprayed with a liquid which is subsequently cured and/or dried to form coating 22, to create such a design. In other instances, depending upon the patient anatomy where device 10 is to be deployed, different and more complex patterns of coating frame 12 might be employed. For instance, rather than an axial segment of frame 12 being coated with coating 22, one latitudinal half of frame 12 might be coated, or still some other combination of non-uniform coating circumferentially about longitudinal axis 18, and axially along frame 12 between ends 30 and 28 might be designed. The detailed enlargement in FIG. 1 illustrates coating 22 upon outer surface 20, and coating 22 ending somewhat abruptly prior to covering 38 so that a relatively short and uncoated axial section of frame 12 extends between coating 22 and covering 38. In a practical implementation strategy, frame 12 may include a wire frame with a plurality of struts 42. Struts 42 might be each irreversibly attached to a plurality of other struts, such as might be achieved by laser cutting frame 12 from a tubular blank of starting material. Struts 42 might also be formed as parts of separate wires woven, braided or otherwise coupled to one another in a manner that will be familiar to those skilled in the field of stent technology. Outer surface 20 will be understood to refer to the collective outer surface of individual struts, not an outer surface of the overall device 10. Accordingly, coating 22 will typically cover exposed surfaces of frame 12 which are inside of device 10, defining an inner lumen thereof and not readily visible in FIG. 1. Stated another way, a plurality of struts 42 will be encased in coating 22.

As noted above, coating 22 serves to anchor device 10 within a body lumen in a patient, and more particularly serves to initiate and promote physiological processes that enable device 10 to be securely retained after a period of residence within the patient's body. To this end, coating 22 includes a sclerosant in a concentration sufficient to incite immune reaction in body tissue so as to trigger new body tissue growth. Those skilled in the art will be familiar with phenomena associated with certain implanted medical devices, where some growth of new body tissue occurs about parts of the implanted device. Such tissue growth is undesired in many cases. Coating 22 may further include a bioremodelable material for promoting ingrowth of the new body tissue about frame 12 so as to anchor device 10 within a body lumen in a patient. It will therefore be appreciated that coating 22 can stimulate or initiate an immunological response in body tissue within a body lumen such as the esophagus, a vein or artery, and then provide for tailoring of the new body tissue growth in a manner promoting retention of an implanted device at a treatment location.

In one practical implementation strategy, the sclerosant may include one or more sodium salts, a sugar such as dextrose or glucose, sodium tetradecyl sulfate, or still another known sclerosant. In the case of sodium tetradecyl sulfate, an amount of sclerosant in coating 22 may be such that a localized concentration of sodium tetradecyl sulfate eluted may be from about 0.01% to about 20% in aqueous solution, preferably in a range from about 1% to about 6%, and most preferably about 3%. Where dextrose is used, a localized concentration of eluted dextrose may be from about 0.01% to about 20% in aqueous solution, preferably from about 5% to about 15%, and most preferably about 12%. Sodium salts used as a sclerosant may be such that a localized concentration is from about 0.01% to about 30% in aqueous solution, preferably from about 5% to about 25%, and most preferably about 23.4%. Also in a practical implementation strategy, the bioremodelable material may be material of biological origin, preferably extracellular matrix (ECM) material, and typically small intestine submucosa (SIS) in so-called particulate or micronized form, and of porcine origin. Such ECM material is readily available from the assignee of the present patent application, for instance.

Coating 22 may further include a matrix material, not to be confused with the ECM, adherent to frame 12, and including one or more of a biodegradable polymer such as PLGA, a plasticizer such as glycerin, or combinations thereof. The milieu of ECM, matrix material, and sclerosant adheres to frame 12, such that one end of device 10/frame 12 becomes anchored while the opposite end remains unanchored within the patient. Frame 12 may be formed from a metallic material such as nitinol, or another superelastic metal alloy. Once implanted, the extracellular matrix material will have the desired effect of promoting ingrowth of new body tissue stimulated via the sclerosant, such that the new body tissue will grow over and around individual elements of the implanted device, commonly in a pattern of new growth congruent with an arrangement of individual elements of the implanted device, in particular a plurality of the struts.

### INDUSTRIAL APPLICABILITY

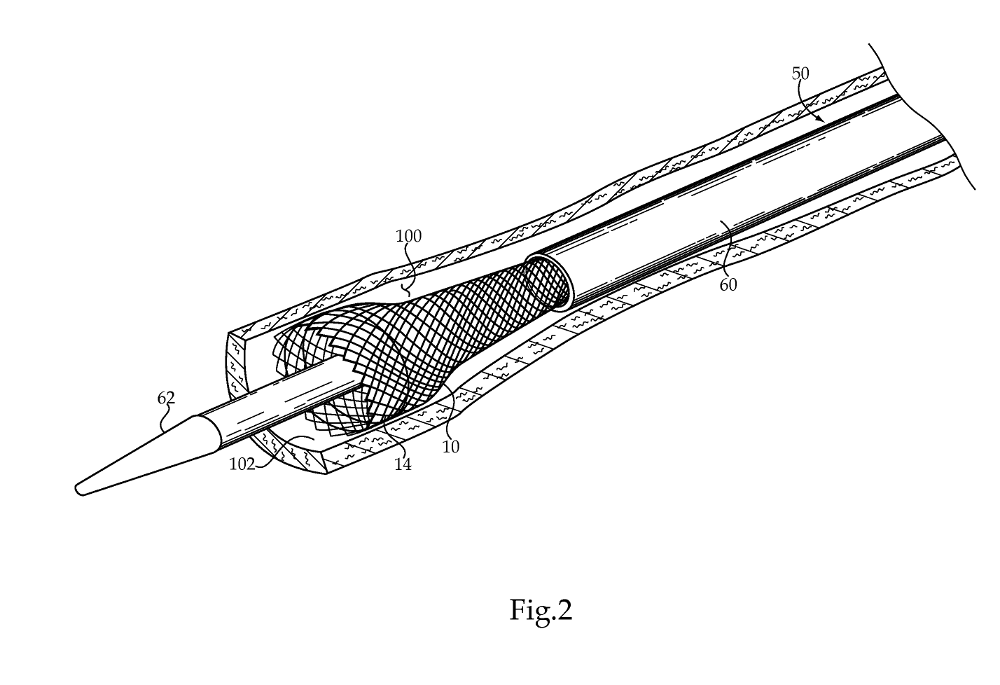

Referring to the drawings now generally, in FIG. 2 device 10 is shown as it might appear having been advanced through a body lumen 100 in a patient to a treatment location. At the treatment location, device 10 is shown in the process of being deployed via a delivery mechanism 50 having a sheath 60 and a guide 62, of generally known type and configuration. As noted above, body lumen 100 might be a gastrointestinal body lumen such as that formed by the patient's esophagus. As also noted above, frame 12 may be formed from a superelastic metal alloy such as nitinol, and thus one example of a so-called shape memory material. Deploying device 10 may include transitioning device 10 from a low profile configuration to an expanded configuration via a shape memory bias of wire frame 12. From the state depicted in FIG. 2, sheath 60 will typically be further withdrawn until a point at which device 10 is fully free of sheath 60 and in its expanded configuration within the patient's body lumen. FIG. 1 illustrates a cord 40 threaded among individual struts of frame 12 at frame end 16. A clinician can use cord 40 to retrieve or reposition device 10, for example prior to device 10 being fully deployed out of sheath 60, in a generally known manner.

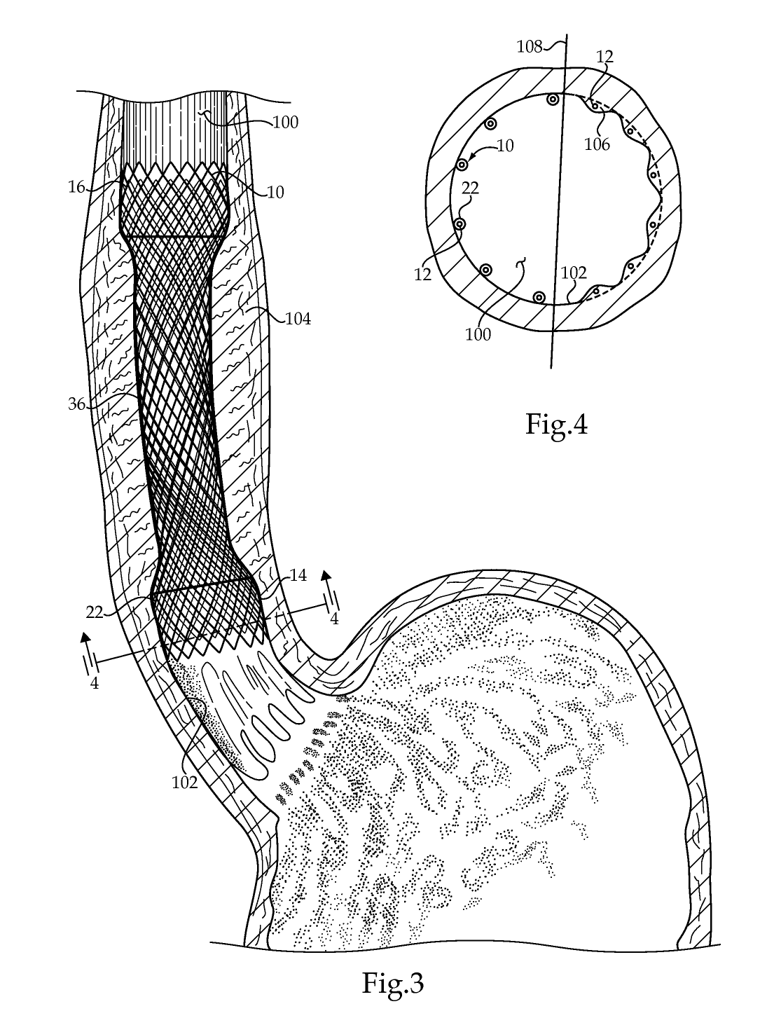

As shown in FIG. 3, once device 10 has been positioned at a treatment location, both of ends 14 and 16 have expanded such that device 10 contacts body tissue forming an inner wall 102 of body lumen 100. Middle section 36 is generally positioned proximate and in contact with abnormal tissue 104 within the body of a patient. In FIG. 4, taken along line 4-4 of FIG. 3, on the left side of a line 108 device 10 is shown as it might appear where initially placed with coating 22 in contact with inner wall 102. In the right half of the FIG. 4 illustration, ingrowth of new body tissue 106 has occurred around frame 12 of device 10. It can be seen that ingrowth of new body tissue 106 has been promoted by coating 22, such that device 10 is anchored by the new body tissue at the treatment location. End 14 may be anchored while end 16 remains unanchored. Promoting of the ingrowth of the new body tissue has occurred such that the new body tissue ingrowth is predominantly about the portion of device 10 that includes coating 22.

As alluded to above, ingrowth of body tissue about implanted devices has been observed for many years. In general, such body tissue ingrowth, often so-called “endothelialization,” is undesired and viewed as an impediment to later extraction of an implanted device. Lacking the recognition that tissue ingrowth can be exploited for device retention, some strategies for device implantation may actually seek to suppress tissue ingrowth while also providing a mechanical means for device retention. Embodiments are contemplated wherein a device according to the present disclosure includes both a coating as contemplated herein and also additional means for retention. In practical implementation strategies, however, device 10 may be free of any such additional retention mechanisms. It will thus be appreciated that the present strategy can be simpler and easier for a clinician to perform, in certain instances presenting less risk of injury or rejection by the patient since the patient's physiology is exploited in retaining the implanted device. While stents, notably gastroesophageal stents, are considered to be a primary application of the present disclosure, other applications are contemplated as discussed herein.

The present description is for illustrative purposes only, and should not be construed to narrow the breadth of the present disclosure in any way. Thus, those skilled in art will appreciate that various modifications might be made to the presently disclosed embodiments without departing from the full and fair scope and spirit of the present disclosure. Other aspects, features and advantages will be apparent upon an examination of the attached drawings and appended claims.

What is claimed is:

1. A method of implanting an endoluminal device in a patient comprising the steps of:
advancing the endoluminal device through a body lumen in the patient to a treatment location;
deploying the endoluminal device at the treatment location such that the endoluminal device contacts body tissue forming an inner wall of the body lumen;
inciting an immune reaction in the body tissue via a sclerosant in a coating of the endoluminal device so as to trigger growth of new body tissue at the treatment location; and
promoting ingrowth of the new body tissue about the endoluminal device via a bioremodelable material in the coating, such that the device is anchored by the new body tissue at the treatment location.

2. The method of claim 1 wherein the bioremodelable material includes an extracellular matrix (ECM) material.

3. The method of claim 2 wherein the ECM is small intestine submucosa particulate in a biodegradable matrix material of the coating.

4. The method of claim 3 wherein the step of promoting includes promoting ingrowth of the new body tissue predominantly about a portion of the endoluminal device that includes the coating.

5. The method of claim 4 wherein the endoluminal device includes a wire frame and the portion that includes the coating includes one end of the wire frame, such that the endoluminal device once implanted is anchored at the one end of the wire frame and an opposite uncoated end of the wire frame is not anchored.

6. The method of claim 5 wherein the wire frame includes a plurality of struts each encased by the coating at the one end of the wire frame, and the step of promoting further including promoting the ingrowth such that the new body tissue forms a pattern congruent with an arrangement of the plurality of struts.

7. The method of claim 5 wherein the wire frame includes a shape memory material, and the step of deploying the endoluminal device includes transitioning the endoluminal device from a low profile configuration to an expanded configuration via a shape memory bias of the wire frame.

8. The method of claim 7 wherein the endoluminal device includes a stent placed via the step of deploying at a treatment location within a gastrointestinal tract of the patient.

9. An implantable endoluminal device comprising:
a frame including a first frame end and a second frame end, and defining a longitudinal axis extending between the first frame end and the second frame end;
the frame further having an outer surface, and a coating upon the outer surface having an axial extent upon the frame less than a full axial length from the first frame end to the second frame end;
the coating including a sclerosant in a concentration sufficient to incite immune reaction in body tissue so as to trigger new body tissue growth, and a bioremodelable material for promoting ingrowth of the new body tissue about the frame so as to anchor the endoluminal device within a body lumen in a patient.

10. The endoluminal device of claim 9 wherein the frame includes a wire frame having a plurality of struts encased in the coating.

11. The endoluminal device of claim 10 wherein the coating includes a matrix material adherent to the wire frame.

12. The endoluminal device of claim 11 wherein the matrix material includes a biodegradable material.

13. The endoluminal device of claim 12 wherein the matrix material includes at least one of a polymer and glycerin.

14. The endoluminal device of claim 10 wherein the wire frame is formed of a superelastic metal alloy.

15. The endoluminal device of claim 10 wherein the bioremodelable material includes small intestine submucosa particulate.

16. The endoluminal device of claim 10 wherein the coating is upon the first frame end and the second frame end is uncoated.

17. The endoluminal device of claim 16 wherein the axial extent of the coating includes a minority of the full axial length.
